(19) United States
(12) Patent Application Publication
(10) Pub. No.: US 2004/0076653 A1
(43) Pub. Date: Apr. 22, 2004

(54) Method for inducing production of fibrous organic tissue

(75) Inventors: Leandra G. Madris, New York, NY (US); Roberto Obando Diaz, Bogota (CO)

Correspondence Address: Henry D. Coleman, 714 Colorado Avenue, Bridgeport, CT, 06605, US

(73) Assignee: Revicel Corporation, New York, NY

(21) Appl. No.: 10/637,791

(22) Filed: Aug. 8, 2003

Related U.S. Application Data
(60) Provisional application No. 60/403,117, filed on Aug. 13, 2002.

(51) Int. Cl. A61K 31/01; A61K 7/00

(52) U.S. Cl. 424/401; 514/762

(57) ABSTRACT

A method and composition for inducing the production of fibrous tissue can be used to treat urinary incontinence or in the creation of cosmetic enhancements such as wrinkle reduction or removal or to treat burn victims by facilitating tissue growth in conjunction with skin grafting. The method includes injecting an effective amount of a pure hydrocarbon petroleum jelly composition into a predetermined location in a mammalian subject.

This application claims the benefit of priority of provisional application No. 60/403,117, filed Aug. 13, 2002.

### FIELD OF THE INVENTION

This invention relates to a method for inducing the generation of fibrous tissue in organic tissues of an individual. The method is useful in the treatment of certain medical conditions, as well as in cosmetic applications. This invention also relates to the use of a harmless compound which induces the production of fibrous tissue in the user's/patient's organic tissues. The product is useful to treat a number of human and animal conditions as well as for cosmetic applications.

### BACKGROUND OF THE INVENTION

It is estimated that 5 percent of the adult population of the world suffers from urinary incontinence. Persons of any age or sex can suffer from urinary incontinence, but the disease tends mainly to affect women. It is estimated that 15 to 20 percent of elderly persons (60 years and over) are incontinent. This is not to suggest that incontinence is a naturally occurring symptom of the aging process. Presently less than half of those suffering from basic urinary incontinence seek medical care. This may be due to the social stigma attached. Given population trends this will become an ever-growing problem needing our attention. The percentage of people with urinary incontinence varies from one country to another. In developing countries, the incidence of urinary incontinence is greater than in other countries.

One of the approved methods of controlling urinary incontinence is the application of collagen injections into the vesicle neck of the urethra for the purpose of shrinking the urinary exit orifice and strengthening the effect of the sphincters. However, within a short period of time it gradually becomes necessary to inject additional collagen. By its very nature, collagen is attacked by the organic system and either absorbed or expelled by the organism, thereby losing its initial effect. Because of the absorption of collagen by the organism, the very use of collagen may in fact exacerbate the condition it is meant to mitigate. However, it has yet to be shown whether repeated injections may actually cause the enlargement of these voids, which would aggravate rather than improve the very condition being treated. Furthermore, the repeated inoculation may very well have scaring effects, which may not be readily acknowledged.

Similar to collagen, there exists silicone and Teflon used for the same pathology, which have resulted in migration problems and unwanted tissue formation. Consequently, these compositions have been inadequate or inadvisable for human use.

### OBJECTS OF THE INVENTION

A general object of the present invention is to provide a method for inducing or generating fibrosis in organic tissues.

It is an additional object of the invention to provide an improved method for treating urinary incontinence. A more specific object of the present invention is to provide a method for treating urinary incontinence, which has a more permanent effect in the patient than treatment using collagen.

Yet an additional object of the present invention is to provide an enhanced product to treat urinary incontinency, with a more long-lasting effect than collagen and with reduced or preferably, no side effects.

Another object of the present invention is to provide a method for producing cosmetic enhancements in an individual, including, but not limited to, the removal of wrinkles in the skin.

Still a further object of the present invention relates to the ability of the present method to enhance and stimulate sexual arousal and orgasm in females by creating fibrotic tissue in and around the female patient's Graefenberg zone (“G-zone”).

These and/or other objects of the present invention will be apparent from the drawings and descriptions herein. Although each object is attained by at least one embodiment of the present invention, it is not necessary that every embodiment of the invention achieve all of the objects of the invention.

### SUMMARY OF THE INVENTION

A method for inducing the production of fibrous tissue comprises, in accordance with the present invention, providing a petroleum jelly composition and injecting an effective amount of the petroleum jelly composition into a predetermined location in a mammalian subject. Petroleum jelly modified compositions according to the present invention represent other aspects of the present invention.

The invention may be used for the cosmetic treatment of wrinkles. In this case, the predetermined location of the injection is into and/or proximate to a wrinkle, which is generally due to natural aging processes, and its surrounding tissues. The injecting of the present composition results in a smoothing of skin in a region about the wrinkle, thus reducing the size and depth of the wrinkle and recovering, to a great extent, the skin smoothness. An effective amount of the composition according to the present invention (preferably, petroleum jelly) is sufficient to at least reduce the size and depth of the wrinkle.

The invention may also be used in the treatment of urinary incontinence. In this case, the predetermined location of the injection is in a urinary excretion pathway, for instance, along a urethra, in proximal urethral submucosa, or in a urinary bladder. The petroleum jelly composition is injected in an amount effective to at least reduce urinary incontinence. The injecting of the petroleum jelly composition may include periurethrally injecting at the vesicle neck level, in the submucosa.

The invention may also be used in grafts, especially skin grafts, muscle grafts, and other kinds of tissue grafts, where depressions or “holes” in the tissue may be resolved through the use of the present methods. In this method, a composition is injected or otherwise delivered to the depression or void in the tissue underneath the skin or other site and allowed to produce a fibrous material at the injection site, resulting in the substantial reduction or elimination of the depression in the tissue.

In an indication related to sexual function in females, a method according to the present invention relates to the composition being injected into or the surrounding tissue of the Gräefenberg or G-zone in a woman's vagina, thus producing fibrotic tissue which will supporting the G-zone and benefit the woman by enhancing the woman's ease of sexual arousal and orgasm.

The fibrosis-inducing composition is preferably a formulation of pure hydrocarbons. Preferably, the petroleum-jelly-containing fibrosis-inducing composition includes hydrocarbon molecules having between about 4 and about 60 carbon atoms. More preferably, the petroleum jelly composition includes hydrocarbon molecules having between about 10 and about 50 carbon atoms only (even more preferably, at least about 12 carbon atoms within this range). Most preferably, the petroleum jelly composition includes hydrocarbon molecules having between about 17 and about 38 carbon atoms only.

In addition or alternatively, other substances with ancillary purposes may be included in the fibrosis-inducing composition. For instance, the composition may additionally incorporate a local anesthetic and/or an anti-inflammatory agent. Any suitable anesthetic or anti-inflammatory agent may be used. Where an injection is being made into a heavily vascularized region, a coagulant may be included in the fibrosis-inducing composition.

An anesthetic may be administered separately to the subject prior to the injecting of the petroleum jelly composition. The administering of the anesthetic may include locally applying the anesthetic to a target injection site.

The effective amount of the petroleum-jelly-containing fibrosis-inducing composition is between about 1 and about 14 ml and more preferably between about 3 ml and about 8 ml.

The injecting of the present composition, preferably a petroleum jelly composition is preferably accomplished using a standard medical hypodermic syringe. However, any kind of syringe may be used in carrying out the method described herein. Other means of injecting a fluidic composition into organic tissues may be alternatively used. Such alternative devices include pressure injectors and injection guns, with or without needles. Where the target tissues are internal to an individual, the delivery of the petroleum jelly composition to the target may be accomplished via another kind of tubular instrument such as a laparoscopic or endoscopic injector or a catheter.

Petroleum jelly, as a preferred composition according to the present invention, exhibits several advantages with respect to its use in a method in accordance with the present invention.

Petroleum jelly is biocompatible with organic human tissues. More particularly, petroleum jelly is inert from the standpoint of histological and immunological reactions. Petroleum jelly is not biodegradable, not does it migrate from an injection site. Accordingly, a single injection can have a lasting effect. This endurance of the composition after injection into a patient facilitates the production of a substantial amount of fibrous tissue. The petroleum jelly does not migrate, degrade, or absorb prior to the formation of new tissues.

Thus, petroleum jelly is not only innocuous to the organism, but achieves desired objectives, without exacerbating the problem whose solution is sought, for instance, a controlled elimination of bodily fluids in the case of urinary incontinence treatment. Furthermore, petroleum jelly is sufficiently inert in the face of the organic metabolic processes and remains firm and stable “permanently” over years with the strength of its “natural” adhesion remaining constant.

The lack of the tendency of petroleum jelly to migrate from an injection site in organic tissues is due at least in part to its viscosity. In contrast, more liquid or less viscous agents can travel from an injection site into other tissues. The lack of migration renders petroleum jelly exempt from complications that other agents might exhibit, stemming from migration and side effects.

Another advantage to the use of petroleum jelly is that it is produced in great abundance and is accordingly cost effective.

Petroleum jelly functions in part to fill up crevices and otherwise displace and/or supplement muscular tissues where needed. Thus, the composition can be used to reduce or “remove” (by filling) skin wrinkles or other tissue deficits, and under some conditions may help to regenerate lost tissue at a faster rate. This regeneration of tissue is invaluable for treating burns and/or other body injuries.

In a preferred composition aspect of the present invention, a petroleum jelly modified composition for injection into a patient and use in the present application is prepared according to the process of:

1. obtaining a sample of Pharmaceutical Degree (USP) petroleum jelly;

2. distilling said petroleum jelly under heat and vacuum to remove a liquid fraction and produce a semisolid fraction of the original sample;

3. heating the semisolid fraction, preferably to a temperature above 100° C., more preferably at least about 120° C.;

4. filtering the semisolid sample; and

5. sterilizing the sample.

In the present aspect of the invention directed to the petroleum jelly modified composition, preferably, the liquid fraction referenced above represents between about 10% and about 35% by weight of the original sample of petroleum jelly, more preferably about 15% to about 35% by weight of the original sample of petroleum jelly, even more preferably about 25% to about 35% by weight of the original sample of petroleum jelly and most preferably about 30% by weight of the original petroleum jelly sample. Optionally and preferably, the sample can be filtered at various stages and autoclaved as well to prepare a more highly purified product. Preferably, the semisolid sample obtained after the vacuum distillation step is heated above 100° C. and filtered using 20-30 micron pore filters, more preferably 22 micron pore filters. Sterilization preferably occurs in an autoclave at a temperature of about 100° C. or more and pressure (preferably, about 15 pounds or more).

### DEFINITIONS

The term “petroleum jelly composition” is used herein to denote a purified mixture of semi-solid, saturated hydrocarbons, mainly of paraffinic nature, obtained from petroleum jelly composition or white jelly “Pharmaceutical Degree (USP)”. The most preferred composition according to the present invention is “petroleum jelly modified composition”. Petroleum jelly is freely obtainable in off-the-shelf preparations generally sold for topical application to skin injuries. Petroleum jelly is a semisolid substance (gel) that does not flow at room temperature. It may be injected into organic tissues via a hypodermic syringe but its natural viscosity at body temperature is so great as to prevent migration from the injection site. In the present application, compositions such as petroleum jelly are used and are clearly preferred. It is preferably a purified mixture of semi-solid, saturated hydrocarbons, mainly of a paraffinic nature. Other compositions which may be used in the present invention include mixtures of C₄-C₆₀ saturated hydrocarbons, more preferably a mixture of saturated C₁₀-C₅₀ hydrocarbons (more preferably at least a C₁₂ hydrocarbon within this range), even more preferably a mixture of saturated C₁₇-C₃₈ hydrocarbons (as exemplified by purified commercial preparations of petroleum jelly), which obtain a favorable viscosity for convenient delivery of the composition into the target tissue of a patient preferably at a temperature of about room temperature (about 20-2520 C.) to slightly higher than body temperature, i.e., about 36° to about 45° C. in the case of human patients, and higher or lower than that temperature range depending upon the type of veterinary application, preferably, about room temperature to about 38° C., even more preferably at about room temperature using a gun. It is noted that when referring to the present compositions, it is the viscosity of the formulation at body temperature and at the temperature of delivery which defines the composition. The present compositions are therefore generally described as mixtures of linear and branch-chained saturated hydrocarbons (“hydrocarbons”) having an effective viscosity which can be conveniently delivered to a site in the tissue of a patient to be treated and after delivery, the hydrocarbon mixture will remain at the site of tissue delivery without any significant migration (substantially no migration or dispersing of material away from the site of injection) for a period of time effective to allow the patient's body to produce fibrous tissue in response to the injected material (preferably, at least a few, ie, at least two, more preferably at least three) days to several weeks,. The newly created fibrous tissue will provide a patient's own natural supplemental “filler” to tissue which has been wrinkled with age, hollowed or damaged as a consequence of physical injury, disease or other conditions.

The term “treatment site” or “target treatment site” refers herein to a site, an area or volume of organic tissues of a patient, subject or individual where the injection of petroleum jelly as described herein is expected to have a desired or beneficial effect. A target treatment site may be as small as a few square or cubic millimeters, or smaller, or as large as a few square or cubic centimeters. Where the treatment is a cosmetic reduction of skin wrinkles, each wrinkle or constellation of wrinkles may be considered a separate treatment site. In the case of skin grafts, damaged skin and related conditions where a natural fibrolytic filler or layer may be desirable to fill deficiencies in body structures, the amount of material may be considerably larger. The administration of the present composition, preferably, petroleum jelly, most preferably petroleum jelly modified composition, to a treatment site, may be effectuated by one or several separate interspaced injections of the petroleum jelly modified composition to the area or volume of the treatment site. It may be administered topically, for example, via skin graph where the patient's own skin may not exist such as may be the case with bum victims. It also may be administered at or near the G-zone in the vagina of a female patient to produce fibrous tissue.

The terms “Gräefenberg spot”, “G-spot” or “G-zone” are used synonymously to describe an area within the vagina of female patients which is believed to swell during sexual arousal and intercourse, leading to orgasm. The G-spot lies directly behind the public bone within the front wall of the vagina. It is usually located about halfway between the back of the public bone and the front of the cervix, along the course of the bladder, where it connects with the urethra. The size and exact location may vary. Unlike the clitoris, the G-zone lies deep within the vaginal wall and a firm pressure is often needed to contact the G spot in its unstimulated state. In the present invention, by providing fibrous tissue within the vagina or other tissue at or near the G-spot, the ability to enhance stimulation of the G-spot is increased, resulting in greater sexual arousal and orgasm by the patient. Thus, the present invention also may be used to increase sexual function in female patients, especially those patients who do not regularly experience orgasm.

The word “injection” and its relatives “inject” and “injecting” are used herein to generally denote a process of depositing a fluidic material (petroleum jelly) in internal organic tissues of a patient. Typically, injection is accomplished via a thin tubular member, such as a hollow needle or catheter. Also, there is preferably a pressure source for applying pressure to the fluidic material to force the fluidic material through the tubular member and into organic tissues of a patient.

The term “fluidic material” is used herein to refer to a non-gaseous material or substance which is capable of fluid flow. Fluidic materials thus particularly encompass liquids and gels such as petroleum jelly.

The term “patient” or “subject” is used to describe a mammal, especially a human, in need of treatment, who is treated with compositions according to the present invention.

The term “viscosity” or “effective viscosity” is used to describe the flowability or density (thickness) of the compositions according to the present invention at the time and temperature of delivery into the patient to be treated. In general, the viscosity of the compositions according to the present invention at the time and temperature of delivery (preferably by injection through a syringe or via a catheter) or treatment is that viscosity which allows for the delivery of effective amounts of the composition to a treatment site within the patient. Thus, an effective viscosity within the context of the present invention is that viscosity of the compositions sufficiently flowable to allow delivery to a treatment site within a patient or subject without allowing substantial transport of the composition away from the injection/treatment site because of excessive flowability. By way of reference and description, and without limiting the present invention in any way, in preferred aspects of the present invention, at the temperature of delivery, compositions according to the present invention will have a viscosity ranging from about 500 centipoise units or less to about 150,000 centipoise units or somewhat more, depending upon the temperature of delivery, pressure to be applied at delivery and the size of the bore through which the composition passes as it is being delivered to a treatment site within the patient or subject. Preferably, the viscosity is about 1500 to about 50,000 centipoise units, more preferably about 2,000 centipoise units to about 30,000 centipoise units, again depending upon the temperature, pressure and bore size of the delivery instrument.

The viscosity of the composition according to the present invention is determined by the number of hydrocarbons included within a composition, the number of carbons within a given hydrocarbon and the relative weight percent such hydrocarbon is included in the final composition and the extent of branching of the hydrocarbons used in the present compositions. As a general rule, as one increases the amount of smaller chain hydrocarbons in a composition and as the branching of any hydrocarbons increases, viscosity will tend to decrease in the final composition. However, higher molecular weight hydrocarbons are normally present in the final composition.

The term “mixture of C₄ to C₆₀ hydrocarbons” and related terms refer to a composition containing at least two hydrocarbons each having from 4 to 60 carbon atoms (or other specified range), preferably at least 10 different (i.e., structurally different, which term may include hydrocarbons having different numbers of carbon atoms, or isomers, including geometric isomers) hydrocarbons within the specified range. Preferably, the mixture contains a varied mixture of hydrocarbons within the stated range. Most preferably, the mixture is a purified form of petroleum jelly.

The term “effective” is used to describe an amount, quality or characteristic (such as viscosity) of a composition according to the present invention within the context of the use of that composition.

### DETAILED DESCRIPTION OF THE PREFERRED EMBODIMENTS

The present invention contemplates the injection of saturated hydrocarbon compositions as described herein, preferably pure hydrocarbon formulations of petroleum jelly into patients to secure a desired result, more particularly, a beneficial medical or cosmetic result. The composition according to the present invention, preferably the petroleum jelly composition naturally has a viscosity sufficiently low to enable injection into organic tissues of a subject via a standard hypodermic syringe. The viscosity is sufficiently high to prevent migration of the injected composition from the injection site.

The viscosity of the petroleum jelly is determined in part by the number of carbon atoms in the constituent molecules, smaller carbon chains being associated with a lower viscosity. Thus, in the present invention, a decrease in the number of carbons in a molecule (molecular weight decrease) or an increase in the branched nature of the hydrocarbon molecule, will result in a lowering of the viscosity of a compound. An increase in the percentage of small molecular weight hydrocarbons in a composition will also generally reduce the overall viscosity of a composition. Likewise, an increase in the number of carbons within a hydrocarbon molecule (molecular weight increase) or an increase in the linear as opposed to branch-chained character of a hydrocarbon mixture, will increase the viscosity of the composition.

The present compositions have a suitable viscosity for delivery to a site in the patient and producing the intended effect at that delivery site in the patient. Most petroleum jelly compositions have a viscosity that is suitable for the present applications and in particular for delivering the compositions to the injection or delivery site within the patient, for example, between about 5 centistokes (cSt) and about 14 cSt at 100° C., preferably less than about 10-11 cSt. The viscosity is low enough to enable injection via a small-diameter lumen but high enough to prevent migration from the injection site. In the event that the viscosity of a selected composition is so high as to render injection difficult, the composition may be heated, within organism-tolerable limits, prior to injection. The temperature of the composition at the time of injection is preferably in a range of about room temperature (about 20-25° C.) to about body temperature to slightly higher than body temperature, i.e., about 36° to about 45° C. in the case of human patients, and higher or lower than that temperature range depending upon the type of veterinary application, preferably, about room temperature to about 38° C. In certain aspects of the present invention, the composition is delivered at about room temperature with a gun.

Generally, at the temperature of delivery, the present compositions will have a viscosity ranging from about 500 centipoise units or less to about 150,000 centipoise units or somewhat more, depending upon the temperature of delivery, pressure to be applied at delivery and the size of the bore through which the composition passes as it is being delivered to a treatment site within the patient or subject. Preferably, the viscosity is about 1500 to about 50,000 centipoise units, more preferably about 2,000 centipoise units to about 30,000 centipoise units, again depending upon the temperature, pressure and bore size of the delivery instrument.

Injection is preferably accomplished using a standard medical hypodermic syringe. However, any kind of syringe may be used in carrying out the method described herein. Other means of injecting a fluidic composition into organic tissues may be alternatively used. Such alternative devices include pressure injectors and injection guns, with or without needles. Where the target tissues are internal to an individual, the delivery of the petroleum jelly composition to the target may be accomplished via another kind of tubular instrument such as a laparoscopic or endoscopic injector or a catheter.

Where an injection of an aliquot of a petroleum jelly composition is expected to cause pain to the patient, for instance, where relatively large amounts of the composition are used as in cosmetic remodeling, an anesthetic may be first administered to the patient. In many instances, treatment of the target injection site with a local anesthetic will be satisfactory.

The injection of compositions according to the present invention and preferably, petroleum jelly, especially petroleum jelly modified composition, as described herein induces the natural production of fibrous tissue in the area of the injection. The fibrous tissue appears in and around the region of the injection. In most cases, the fibrous tissue formed is permanent. It is not necessary for the patient to undergo repeated injections at periodic, intervals. However, several follow-up injections may be desirable in certain cases, for example, where a facial contour is being remodeled. Several injection treatments over time will result in a more gradual change in the appearance of the individual, which may be desired, for instance, to minimize detection by others.

After treatment as described herein, each patient's progress is followed intently or intensively for a few days. Generally, patients respond well to the procedure. Each patient's progress may be followed with less intensive monitoring for a longer period, for example, two years, to ascertain that the treatment is permanent.

A predetermined effective amount of the composition according to the present invention (preferably, petroleum jelly) is injected into muscle tissue or connective tissue or into interstitial spaces between muscle tissue and connective tissue, depending on the result desired. Ideally, there is no diffusion of the injected composition from the injection site

Generally, the total amount of the composition according to the present invention (preferably, petroleum jelly) injected into a target treatment site is between about 0.5 ml and about 14 ml, preferably at least about 1 ml within this range. More preferably, the total injected amount is between about 3 ml and 8 ml. The petroleum jelly may be injected as a single aliquot or may be injected as several smaller amounts at locations spaced from one another within the area or volume of the treatment site. Small amounts of the present compositions (preferably, petroleum jelly) are typically used in the treatment of fine facial wrinkles, for instance, about the eyes, whereas larger amounts are typically required where the treatment is for a medical condition (e.g., incontinence), for example, between about 4 and 7 ml. In the case of treatment of G-zones to promote sexual function in female patients, an amount of composition ranging from as little as 0.5 ml up to about 3-5 ml preferably may be used.

A preferred petroleum jelly for use in the present method is a pharmaceutical-grade white petroleum jelly having the tradename ROD-3 available from Revicell Corporation, 156 Fifth Avenue, New York, N.Y. 10010. ROD-3 has a density in a range between 0.815 and 0.880 kg/L at 60° C. (USP XXIII), a melting range between 38° and 60° C. (USP XXIII), an acidity/alkalinity of neutral to litmus (USP XXIII, BP 1999), a congealing point of 63° C. (exp.), a consistency of 100 to 300 (USP XXIII) at 25° C., a viscosity of 10.7 cSt at 100° C., a UV absorbance at 290 nm of no more than 0.5 (BP 1999), and a flash point of 1900° C. (COC). It has a residue on ignition of no more than 0.05% (USP XXIII). The composition is insoluble in water (USP XXIII, BP 1999) and soluble in chloroform, ether and petroleum spirits (BP 1999). The color is white, with no fluorescence (USP XXIII, pp. 1196). The composition preferably exhibits an absence of polycyclic hydrocarbons.

The preparation of ROD-3, the preferred composition according to the present invention, proceeds as follows. This product, the preparation as described, was used in the experimental section of the present application which follows. A base raw material of 1000 ml. of White Jelly “Pharmaceutical Grade (USP)”, previously controlled by USP or BP methods is poured into a 2000 ml precipitate vessel and covered. The contents are autoclaved at 120° C. and 15 LB pressure for a period of 30 minutes. The contents are then filtered through Wattman No. 11 filter paper. The filtrate is then poured into a 5 liter round bottom flask. The flask is then attached to a vacuum distillation apparatus, with the joints vacuum tight. A vacuum pump (attached) is then switched on and the vacuum pump is allowed to run without heating the petroleum jelly base material in order to expel air from the vacuum distillation apparatus. The round bottom flask is thereafter slowly heated, while maintaining a constant vacuum. The heat is raised slowly such that material has not distilled within an hour.

When the temperature reaches 190° C., heating becomes quite vigorous and the first distilled drops start to appear. Vigorous heating continues until the distilled material represents approximately 300 ml, at a temperature of about 240° C. Once the required volume is obtained at the indicated temperature, vacuum continues at least for another hour or until the system has reached room temperature. Once room temperature is reached, the vacuum is slowly released and the round bottom flask is removed from the distillation apparatus. The liquid fraction, which was distilled off, is discarded. The round bottom flask containing semisolid product is properly covered up to prevent humidity from entering the flask and the material is again autoclaved at 120° C. and 15 LB pressure for 30 minutes to ensure total heating and fusion. Once the product is out of the autoclave, the product is filtered using 22 Micron Millipore filter. The product is thereafter dosed in usable fractions (for example, 250 ml) into autoclave containers under laminar flow hood. The containers with material are thereafter autoclaved/sterilized for one hour at 120° C. and 15 LB pressure. Storage is at 4-8° C. Dosing is in appropriate aliquots (for example 3 cc) in syringes under laminar flow hood.

Thus, the above-prepared ROD-3 is, from the physical-chemical point of view, a viscous mixture of organic molecules that resemble each other, which are stable chemically and biologically. It is insoluble in water, alcohol, bases, and inorganic solvents; soluble in organic solvents such as ether, chloroform, and carbon sulfide. It is practically inert in the presence of such powerful chemical agents as acids and mineral and organic bases. There is no evidence of any reactions under biological conditions with either proteins or enzymes.

Petroleum jelly (raw material) has an average composition identified by Table 1 below:

TABLE 1

| HYDROCARBON | RAW MATERIAL | | | |
|---|---|---|---|---|
| NUMBER OF | % | % NON- | % | % |
| CARBON ATOMS | LINEAR | LINEAR | AROMATIC | OTHERS |
| 17 | 0.4 | | | |
| 18 | 0.8 | | | |
| 19 | 1.1 | | | |
| 20 | 2.1 | | | |
| 21 | 2.7 | 0.4 | | |
| 22 | 3.1 | 1.9 | | |
| 23 | 3.9 | 0.3 | | |
| 24 | 2.8 | 3.4 | | |
| 25 | 3. | | | |
| 26 | 3.4 | 0.3 | | |
| 27 | 3.5 | 0.2 | | |
| 28 | 4.3 | 0.3 | | |
| 29 | 6 | | | |
| 30 | 6.2 | 2.3 | | |
| 31 | 6.4 | 0.1 | | |
| 32 | 6.1 | | | |
| 33 | 4.6 | 0.3 | | |
| 34 | 4.2 | 0.8 | | |
| 35 | 3.2 | | | |
| 36 | 2.5 | | | |
| 37 | 2.1 | | | |
| 38 | 1 | | | |
| TOTAL | 73.4 | 10.2 | 0 | 16.4 |

A typical petroleum jelly composition sold under the tradename ROD-3 has the composition set forth in the following Table 2, below. The detailed components were determined by HPLC and mass spectroscopy.

TABLE 2

| ATOMS | | | | | |
|---|---|---|---|---|---|
| OF | | % | % NON- | % | % |
| CARBON | % LINEAR | STDESV | LINEAR | STDESV | OTHERS |
| 17 | | | | | |
| 18 | 0.20 | | 0.90 | | |
| 19 | | | | | |
| 20 | | | | | |
| 21 | 0.20 | | | | |
| 22 | 0.40 | | | | |
| 23 | 0.93 | 0.43 | | | |
| 24 | 1.78 | 0.62 | 0.87 | 0.81 | |
| 25 | 2.92 | 0.55 | 0.33 | 0.12 | |
| 26 | 4.17 | 0.64 | 1.32 | 0.98 | |
| 27 | 5.15 | 0.64 | 1.70 | 1.04 | |
| 28 | 6.65 | 0.76 | 1.85 | 0.95 | |
| 29 | 8.05 | 0.61 | 0.58 | 0.08 | |
| 30 | 9.37 | 0.62 | 0.80 | 0.42 | |
| 31 | 9.42 | 0.45 | 1.20 | 0.36 | |
| 32 | 8.88 | 0.41 | 0.45 | 0.19 | |
| 33 | 6.92 | 0.50 | 1.10 | 0.63 | |
| 34 | 6.45 | 0.48 | 0.92 | 0.96 | |
| 35 | 4.68 | 0.81 | 1.12 | 0.90 | |
| 36 | 3.30 | 0.77 | 1.22 | 0.59 | |
| 37 | 2.32 | 0.73 | 1.57 | 1.10 | |
| 38 | 1.52 | 0.94 | 3.00 | | |
| TOTAL | 91.92 | | 12.58 | | 5.50 |

In Table 2, the heading “% others” means saturated hydrocarbon percent which are not datable by physicochemical methods and the heading “%STDESV” refers to the corresponding standard deviation of the method.

In a series of experimental investigations discussed hereinbelow, ROD-3, prepared above, was injected (subcutaneously, intramuscularly, and submuscularly) into laboratory animals, namely, mice, hamsters, and dogs, for the purpose of studying their tissue and systemic reactions to ROD-3. The investigations revealed that under application conditions, ROD-3 is an inert substance, does not migrate within the body, will not decompose in the organism, is not toxic, even at high concentrations, does not produce granulomas or oncogenic cells, causes insignificant inflammation, and within days becomes safely incorporated yet isolated within the organism by a fibrous layer that encapsulates it until stabilized. The organism treats ROD-3 as naturally belonging without any reaction common to the introduction or presence of a foreign body. Injections into the urethra and bladders of dogs showed excellent formation of fibroids and a connective layer with excellent mechanical adhesion.

### Experiment I

Under a completely random experimental design, with factorial adjustment, in which the factors considered were dose, reaction time, lot of ROD-3 used, and sex of the animal 200 mice of both sexes, of SWISS and BALB/C stock, weighing between 30 g and 40 g and 4 months old, were inoculated subcutaneously with doses of 0.03 ml, 0.06 ml, and 0.12 ml (equivalent to 60 ml, 120 ml and 240 ml in adult humans weighing from 60 to 80 kg respectively), in each of their glutei, at a room temperature of 20° C. under aseptic conditions, with the purpose of observing the macro and microscopic action of the product and reaction of the tissues adjacent to it. Biopsies were taken of tissues around each inoculum at times varying between 15 and 150 days, after the inoculation, and histopathologically analyzed. The hypotheses posed were as follows:

H (1): The organism encapsulates ROD-3 by means of a fibrous layer that solidifies with time.

H (V: The inflammation produced by ROD-3 tends to diminish with time.

H (4): The organism does not generate granulomas in response to the presence of ROD-3.

H (V: The organism does not generate oncogenic cells in the presence of ROD-3.

### Experiment II

Under a completely random statistical design, 20 mice of the SWISS stock weighing between 30 and 40 g and 4 months old, were inoculated intraperitoneally with 0.05 ml of ROD-3 (equivalent to 120 ml of ROD-3 in an adult human weighing 60 to 80 kg.) for the purpose of evaluating damage in the liver and kidney due to the toxicity of ROD-3. At 120 days after inoculation, biopsies were made of these organs, which were taken for histopathological analysis. Blood samples were simultaneously drawn by means of cardiac puncture of the animals.

### Experiment III

Under a completely random statistical design, with factorial adjustment, in which the factors considered were dose, reaction time, and sex of the animal, 48 hamsters of both sexes, one month old and weighing approximately 200 grams, were inoculated subcutaneously and intramuscularly with a dose of 0.05 ml and 0.1 ml of ROD-3 (equivalent to 15 ml and 30 ml in an adult human weighing 60 kg.), in each of the glutei, at room temperature (20° C.), with the purpose of macroscopically and microscopically observing the action of the product on the tissues adjacent to it. Biopsies were taken of the tissues around each inoculum at times varying between 15 and 150 days after inoculation and histopathologically analyzed. Blood samples were simultaneously taken by cardiac puncture for analysis of changes in the leucocytes, hematocrit, and transaminases. The hypotheses posed were as follows:

H(1): The organism encapsulates ROD-3 by means of a fibrous layer that solidifies with time.

H(2): The inflammation produced by ROD-3 tends to diminish with time.

H(3): The organism does not generate granulomas in response to the presence of ROD-3.

H(4): The organism does not generate oncogenic cells in the presence of ROD-3.

H(5): The leucocytes do not change in the presence of ROD-3.

H(6): The hematocrit does not change in the presence ROD-3.

H(7): The transaminase content does not change in response to the presence of ROD-3.

H(8): The liver and kidney tissues of the animals inoculated with ROD-3 do not suffer damage.

### Experiment IV

1. Under a completely random experimental design, with factorial adjustment, in which the factors considered were reaction time of ROD-3 and sex of the animal, 36 mice of both sexes, of SWISS and BALB/C stock weighing between 30 and 40 grams and 4 months old, were inoculated subcutaneously at room temperature with a dose of ROD-3 labelled with C¹⁴ (equivalent to 40 ml in an adult human weighing between 60 and 80 kg.) with the aim of valuing its distribution in the liver, kidney, and spleen of the animal. At times varying between 1 and 90 days alkaline digestions were made, at 120° C. and 15 lbs of pressure, from the liver, kidney, and spleen of each of animals, and by means of liquid flashing spectroscopy on aliquot parts of 1 ml of tissue solution, the activity present in each organ stemming from the inoculate of ROD-3 labelled with C¹⁴ was determined. The animals inoculated with ROD-3, treated with formaldehyde, were placed on radiographic plates for visualization, by the technique of autoradiography, the location of traces of the product, which had migrated to different organs. The hypotheses posed were:

H (1): ROD-3 does not move from the inoculation site.

H (2): ROD-3 does not migrate to the animal's organs.

2. Under a completely random experimental design, with factorial adjustment, 19 hamsters of both sexes, 2 months old and weighing approximately 300 g, of both sexes, were inoculated subcutaneously and intramuscularly at room temperature with a dose of 0.02 ml of ROD-3 (equivalent to 6 ml in an adult human weighing 60 kg) with the aim of valuing its distribution in different organs of the animal. At times varying between 15 and 90 days alkaline digestions were made, at 120° C. and 15 lbs of pressure, of tissue samples from the liver, kidney, and spleen of e4ch of the animals, and by means of liquid flashing spectroscopy on aliquot parts of 1 ml of tissue solution, the activity present in each organ stemming from the inoculate of ROD-3 was determined. The animals inoculated with ROD-3, treated with formaldehyde, were placed on radiographic plates for visualization, by the technique of autoradiography, the location of traces of the product that had migrated to different organs. The hypotheses posed were:

H (1): ROD-3 does not move from the inoculation site.

H (2): ROD-3 does not migrate to the animal's organs.

### Experiment V

Under a completely random experimental design, 21 dogs of different races, sexes, ages, and origins, were inoculated subcutaneously, intramuscularly, and in the vesical mucosa with 1-ml doses of ROD-3. At times of 48 days for vesical mucosa and between 15 and 75 days after subcutaneous inoculation, samples were taken of tissues around the product, which were histopathologically analyzed. The hypotheses posed were:

H (1): The organism encapsulates ROD-3 by means of a fibrosis that consolidates with time.

H (2): The inflammation produced by ROD-3 tends to diminish with time.

H (3): The organism does not generate granulomas in response to the presence of ROD-3.

H (4): The organism does not generate oncogenic cells in response to the presence of ROD-3.

H (5): The leucocytes do not change in the presence of ROD-3.

H (6): The hematocrit does not change in the presence of ROD-3.

H (7): The transaminase content does not change in response to the presence of ROD-3.

### Experiment VI

In this experiment, fifty mice of the hybrid stock B₆D₂F₁, were, subcutaneously inoculated with doses of 0.01 ml of ROD-3, with the purpose of evaluating the possible formation of cancerous cells around the inoculum, in the liver, kidneys, and spleen. Simultaneously, cultures were made of cells from the medulla osea in order to evaluate possible alterations at the cellular level. The first results, the product of the sacrifice of 25 mice at seven months after inoculation, revealed no cellular changes.

### Results and Analysis

In the present work, the following were defined as cellular responses of tissues to the presence of ROD-3 in contact with them: 1) the presence of inflammation, 2) the formation fibrosis, 3) the formation of neoplastic cells, and 4) the formation of granulation tissue.

From an organic viewpoint, the following variables were defined: 1) changes in the leucocytes, 2) changes in the hematocrit, and 3) changes in the normal values of blood transaminases.

### Experiment 1

The sources of variation considered in the analysis of the organic action of ROD-3 on tissues being inoculated subcutaneously in mice of SWISS and BALB/C stock were basically four in number. Firstly, the actions of any substance within a living organism are directly correlated with its concentration or dose (this is considered a source of variation). In the experiment, the variable doses of the compound represent concentrations on the order of 60-140 ml of the product in an adult human with an average weight between 60 and 80 kg. For the proposed objective, these doses are very high, since for the control of urinary incompetence, smaller doses are used, for example, doses of 3.5 to 8 ml of total collagen at the periurethral level or the neck of the vesicle. The lethal dose of 50 has to be much greater than the applied maximum dose in this experiment, and it was observed in the course of the experiment that no mice died from this cause. A patient would be administered a dosage greater than 240 ml of this product with great difficulty, and only under very specific conditions.

The second source of variation considered was the sex of the animals, which was known throughout the experiment.

The third source of variation in this experiment is the production lots of ROD-3. Four separate lots were applied with production times of 23, 18, 12, and 6 months, all previously controlled microbiologically and chemically through bacteria cultures, pyrogen analysis, and analysis of chemical-chemical stability using such analytical methods as gas chromatography, IR spectroscopy, mass spectroscopy, and colliquative properties.

Finally, the reaction time of ROD-3 on the tissues was considered the fourth source of variation in the experiment.

Table 3 below presents a summary of the significances of sources of variation in the variance analysis of responses of the subcutaneous tissues to the inoculation of ROD-3 in mice of SWISS stock. Table 3 shows the results of varying some of the variables, including dose, lot, sex, and time, with respect to fibrosis, inflammation, formulation of granulation tissue (granulomas), and, the production of oncogenic cells (neoplasia) produced by ROD-3 when subcutaneously inoculated in mice of the SWISS stock. It is observed that there are highly significant differences (***; P<0.0001) for the variables of time and sex, but no statistically significant differences were observed for the variables of dose and lot.

TABLE 3

| VARIA- | INFLAM- | | | |
|---|---|---|---|---|
| BLES | MATION | FIBROSIS | NEOPLASIA | GRANULOMAS |
| DOSAGE | NS | NS | 0 | 0 |
| TIME | *** | *** | 0 | 0 |
| LOT | NS | NS | 0 | 0 |
| SEX | *** | *** | 0 | 0 |

Table 4 below is a summary of the significances of sources of variation in the variance analysis of responses of the subcutaneous tissues to the inoculation of ROD-3 in mice of BALB/C stock and presents the results of the analysis of variance (ANAVA) of the variables of dose, lot, sex, and time with respect to fibrosis, inflammation, formation of granulation tissues (granulomas), and the production of oncogenic cells (neoplasms) produced by ROD-3 when inoculated subcutaneously in mice of BALB/C stock. It is observed that there are highly significant differences (***: P<0.001) for the variables of time and sex whereas no statistically significant differences were observed for the variables of dosage and lot.

TABLE 4

| VARIA- | INFLAM- | | | |
|---|---|---|---|---|
| BLES | MATION | FIBROSIS | NEOPLASIA | GRANULOMAS |
| DOSAGE | NS | NS | 0 | 0 |
| TIME | *** | *** | 0 | 0 |
| LOT | NS | NS | 0 | 0 |
| SEX | *** | *** | 0 | 0 |

Tables 3 and 4 agree in showing that highly significant differences in the inflammation and fibrosis responses exist only for the variables sex and time. FIG. 4 shows the graph of the tendencies of the animals' responses to the action of ROD-3 injected subcutaneously. In general terms, a good connective reaction appears in a majority of the individuals, with a greater tendency for it to be a moderate reaction whose fibrosis encapsulato.s and isolates the product. The presence has likewise been observed of an inflammatory, reaction with a greater tendency to be mild. The fact of there not being any statistically significant differences for the stocks in their reactions with respect to the variables of dose and lot shows that the product does indeed have no pharmacological action on the organisms, as was to be expected, given the physical/chemical properties of ROD-3 and that the product is chemically stable in the course of at least two years under the packaging conditions in which it was used. The proposed method of packaging is in glass syringes under a laminar flux bell, preserved in hermetically sealed cases and in individual doses of 1 ml. Out of a total of 884 histopathologies of, tissue from mice of SWISS and BALB/C stock, not a single case of the presence of oncogenic cells or granulomas was observed.

### Experiment II

Table No. 5 shows the average values of the histopathologic responses of the liver and kidney in mice of the SWISS stock, 120 days after being inoculated intraperitoneally with ROD-3. The intraperitoneal zone is the zone of maximum absorption and histopathologies of the organs showed the nonexistence of damage to the liver and kidney.

TABLE 5

| | INFLAM- | | |
|---|---|---|---|
| HISTOPATHOLOGY | MATION | NEOPLASIA | GRANULOMAS |
| LIVER | 0 | 0 | 0 |
| KIDNEY | 0 | 0 | 0 |

The values of leucocytes, hematocrits, and transaminases did not undergo any alteration in the inoculated mice as compared to the controls. Macroscopically, one capsule of the product may be observed, white, ovoid in shape, located alongside the organs, separated from them by a transparent layer of connective tissue.

### Experiment III

Table No. 6 shows the results of the variance analysis (ANAVA) of the variables of dose, lot, sex, and time with respect to fibrosis, inflammation, formation of granulation tissue, and production of oncogenic cells (neoplasia) produced by ROD-3 when inoculated intramuscularly in hamsters. It is observed that there are no highly significant differences (***: P<0.001) for the variables of time and sex. Given that the responses considered in the experiment did not present significant differences regarding the factors of dose and lot in mice of SWISS and BALB/C stock, these were not considered to be lacking in significance for this experiment.

TABLE 6

| | INFLAM- | | | |
|---|---|---|---|---|
| VARIABLE | MATION | FIBROSIS | NEOPLASIA | GRANULOMAS |
| TIME | NS | NS | 0 | 0 |
| SEX | NS | NS | 0 | 0 |

With respect to general responses over time of the reactions adjacent to the inocula in hamsters, neither oncogenic cells nor granulation tissues were observed. There is a good fibrotic reaction and a strong inflammation that becomes stabilized with time. No differences appeared between the control values of transaminases, leucocytes, and hematocrit and those obtained in the animals after inoculation.

### Experiment IV

Table 7 displays the results (based upon radioactivity) of the ROD-3 absorbed by the liver, kidneys, and spleen of mice of SWISS and BALB/C stocks, subcutaneously inoculated with DPM units of ROD-3. Table 6 shows that there has been no absorption of ROD-3 in the animals' organs and that the activity of the inoculum, expressed in DPM of ROD-3 under application conditions.

TABLE 7

| ORGAN | TIME (DAYS) | 1 | 3 | 6 | 14 | 21 | 29 | 59 | 91 |
|---|---|---|---|---|---|---|---|---|---|
| LIVER | SWISS BALB/C | 0 | 0 | 0 | 0 | 0 | 0 | 0 | 0 |
| KIDNEY | SWISS BALB/C | 0 | 0 | 0 | 0 | 0 | 0 | 0 | 0 |
| SPLEEN | SWISS BALB/C | 0 | 0 | 0 | 0 | 0 | 0 | 0 | 0 |

### Experiment V

Table 8 shows the results of variance analysis with respect to inflammation, fibirosis, the presence of oncogenic cells and granulation tissue in dogs of different ages and.races inoculated with ROD-3 upon the vesicle mucosa and subcutaneous tissues.

TABLE 8

| | INFLAM- | | | |
|---|---|---|---|---|
| VARIABLE | MATION | FIBROSIS | NEOPLASIA | GRANULOMAS |
| TIME | NS | NS | 0 | 0 |
| SEX | NS | NS | 0 | 0 |

There are no significant differences (NS) with respect to the variables considered when tissue from the vesicle mucosa. and the bladder were histopathologically analyzed. A marked tendency to the formation of strong fibrosis may be observed, accompanied by an inflammatory reaction with the same characteristics. At the present time, 16 dogs, whose weight varies between 15 and 30 kg., have been subcutaneously inoculated, in the vesicle mucosa and periurethrally, with the purpose of making a more rigorous study of the behavior of ROD-3 in dogs. This study furthermore includes the possible changes in the composition of blood and, in part, of urine after inoculation in order to evaluate the systemic behavior of the petroleum jelly product.

The experiment on the fifty mice of the hybrid stock B₆D₂F₁, inoculated with doses of 0.01 ml of ROD-3, for the purpose of evaluating possible formation of cancerous cells around the inoculum, in the liver, kidneys, and spleen evidenced that the product, produced no cellular changes after the sacrifice of 25 mice at seven months after inoculation.

Although the invention has been described in terms of particular embodiments and applications, one of ordinary skill in the art, in light of this teaching, can generate additional embodiments and modifications without departing from the spirit of or exceeding the scope of the claimed invention. For instance, the present compositions may be used in cosmetic applications other than the elimination or reduction of wrinkles. Larger amounts of the present composition or higher numbers of injections may be used to change the shape or contour of a skin surface. This modeling is particularly effective in the face, to effect subtle and not-so-subtle changes in a person's visage or profile. The present compositions may be injected into the lips to increase the size and shape thereof. In men, injections may be made into the chin area or jaw to enhance the impression of strength or into the supra orbital torus to provide a more rugged look. In both women and men, the cheekbones may be accentuated. Other areas of possible cosmetic enhancements include the elbows, the knees, and the backs of the hands. Basically, the injection of a petroleum jelly composition may be at any location where permanent structural supportive tissue may provide a benefit. Injection into mammary glands is contraindicated, except possibly to generate a nipple in breast reconstruction surgery after a mastectomy. Use of the compositions in conjunction with grafting of skin or other tissue represents another embodiment of the present invention.

In all cases, the number of injections, the amounts of the injections and the locations of the injections are predetermined upon consultation with an appropriate professional, such as a plastic surgeon, a urologist, etc., as warranted.

It is to be noted that an injected composition may include components other than petroleum jelly hydrocarbons. For instance, the composition may additionally incorporate a local anesthetic and/or an anti-inflammatory agent. Where an injection is being made into a heavily vascularized region, a coagulant inducing agent may be included in the fibrosisinducing composition.

Accordingly, it is to be understood that the drawings and descriptions herein are proffered by way of example to facilitate comprehension of the invention and should not be construed to limit the scope thereof.

What is claimed is:

1. A method for inducing the production of fibrous tissue in a mammalian subject, comprising: providing a composition consisting essentially of a mixture of saturated hydrocarbons having between 4 and 60 carbon atoms, said composition exhibiting an effective viscosity for delivery to a predetermined delivery site in said subject to be treated; and delivering an effective amount of said composition to said site in said subject.

2. The method defined in claim 1 wherein said composition includes at least 10 different saturated hydrocarbons having between 10 and 50 carbon atoms only.

3. The method defined in claim 1 wherein said composition includes at least 10 different saturated hydrocarbons having between 12 and 50 carbon atoms.

4. The method defined in claim 1 wherein said composition includes saturated hydrocarbons having between 17 and 38 carbon atoms only.

5. The method defined in claim 1 wherein said composition is a pure hydrocarbon formulation of petroleum jelly.

6. The method defined in claim 1 wherein said predetermined delivery site is in a region including organic tissues taken from the group consisting of muscle tissue, dermal, and connective tissue.

7. The method defined in claim 6 wherein said predetermined delivery site is proximate to an age wrinkle, the injecting of said composition resulting in a smoothing of skin in a region about said wrinkle, said effective amount of said petroleum jelly composition being sufficient to at least reduce said wrinkle.

8. The method defined in claim 1 wherein said predetermined delivery site is in a urinary excretion pathway, said effective amount of said composition being sufficient to at least reduce urinary incontinence.

9. The method defined in claim 8 wherein said predetermined delivery site is at proximal urethral submucosa.

10. The method defined in claim 9 wherein the injecting of said composition includes periurethrally injecting at the vesicle neck level, in the submucosa.

11. The method defined in claim 8 wherein said predetermined delivery site is taken from the group consisting of a urethra and a urinary bladder.

12. The method defined in claim 1, further comprising administering an anesthetic to said mammalian subject prior to the injecting of said composition.

13. The method defined in claim 12 wherein the administering of said anesthetic includes locally applying said anesthetic to a target injection site.

14. The method defined in claim 1 wherein said effective amount is between about 1 and about 14 ml.

15. The method defined in claim 14 wherein said effective amount is between about 3 ml and about 8 ml.

16. The method defined in claim 1 wherein the delivery of said composition comprises injecting said subject with said composition using a syringe.

17. A method for treating urinary incontinence in a mammalian subject comprising injecting an effective amount of a composition consisting essentially of a mixture of C₄ to C₆₀ hydrocarbons, said composition exhibiting a viscosity effective for delivery into a predetermined location along a urinary pathway of said subject.

18. The method defined in claim 17 wherein said predetermined location is at proximal urethral submucosa.

19. The method defined in claim 18 wherein the injecting of said petroleum jelly composition includes periurethrally injecting at the vesicle neck level, in the submucosa.

20. The method defined in claim 17 wherein said composition comprises a mixture of hydrocarbons selected from the group consisting of C₁₀ to C₅₀ saturated hydrocarbons.

21. The method defined in claim 17 wherein said composition comprises a mixture of hydrocarbons selected from the group consisting of C₁₂ to C₅₀ saturated hydrocarbons.

22. The method defined in claim 17 wherein said composition comprises a mixture of C₁₇ to C₃₈ saturated hydrocarbons.

23. The method defined in claim 17 wherein said composition is a pure hydrocarbon formulation of petroleum jelly.

24. The method defined in claim 17 wherein said predetermined location is taken from the group consisting of a urethra and a urinary bladder.

25. The method defined in claim 17 wherein said effective amount is between about 1 and about 14 ml.

26. The method defined in claim 17 wherein the injecting of said petroleum jelly composition includes using a syringe.

27. A method for producing cosmetic enhancements in a patient, comprising injecting an effective amount of a composition consisting essentially of a mixture of hydrocarbons selected from the group consisting of C₄ to C₆₀ saturated hydrocarbons, said composition exhibiting an effective viscosity for delivery to a predetermined delivery site in said subject to be treated; and delivering an effective amount of said composition into organic tissues of a patient in a predetermined location of a desired cosmetic modification.

28. The method defined in claim 27 wherein said composition comprises a mixture of hydrocarbons selected from the group consisting of C₁₇ to C₃₈ saturated hydrocarbons.

29. The method defined in claim 27 wherein said composition is a pure saturated hydrocarbon formulation.

30. The method defined in claim 27 wherein said effective amount is between about 1 and about 14 ml.

31. The method defined in claim 27 wherein the injecting of said petroleum jelly composition includes using a syringe.

32. A method of effecting a tissue graft in a patient comprising delivering to a depression or void in the tissue of said patient an effective amount of a composition consisting essentially of a mixture of saturated hydrocarbons selected from the group consisting of C₄ to C₆₀ saturated hydrocarbons, said composition exhibiting a viscosity effective for delivery to a predetermined delivery site in said subject to be treated; and allowing said composition to produce a fibrous material at the injection site, resulting in the substantial reduction or elimination of the depression or void in the tissue of said patient.

33. The method defined in claim 32 wherein said composition comprises a mixture of hydrocarbons selected from the group consisting of C₁₇ to C₃₈ saturated hydrocarbons.

34. The method defined in claim 32 wherein said composition is a pure saturated hydrocarbon formulation.

35. The method defined in claim 32 wherein said effective amount is between about 1 and about 14 ml.

36. The method defined in claim 32 wherein the injecting of said petroleum jelly composition includes using a syringe.

37. A sterilized petroleum jelly modified composition adapted for injection into a patient consisting essentially of a semisolid composition produced according to the process of:
1. obtaining an original sample of pharmaceutical grade (USP) petroleum jelly;
2. distilling said petroleum jelly under conditions of heat and vacuum to remove a liquid fraction and produce a semisolid fraction of the original sample;
3. heating the semisolid fraction;
4. filtering the semisolid fraction; and
5. sterilizing the semisolid.

38. The composition according to claim 37 wherein said liquid fraction comprises about 10% to about 35% by weight of said sample.

39. The composition according to claim 37 wherein said liquid fraction comprises about 25% to about 35% by weight of said sample.

40. The composition according to claim 37 wherein said liquid fraction comprises about 30% by weight of said sample.

41. The composition according to claim 38 wherein said semisolid fraction is heated to a temperature of at least about 100° C.

42. The composition according to claim 38 wherein said semisolid fraction is heated to a temperature of at least about 120° C.

43. The composition according to claim 38 wherein said filtering step utilizes a 20-30 micron pore size filter.

44. The composition according to claim 38 wherein said filtering step utilizes a 22 micron pore size filter.

45. The composition according to claim 38 wherein said semisolid fraction is sterilized under heat and pressure.

46. An injectable petroleum jelly modified composition obtained from the fractional distillation of pharmaceutical grade petroleum jelly after removing a liquid fraction, said composition exhibiting the following physico-chemical characteristics:
1. is a white, viscous jellied mass;
2. is lightly odourless when rubbed on the skin;
3. is water insoluble, but is soluble in chloroform, in ether and in petroleum spirit;
4. exhibits no fluorescence;
5. has a density in a range between about 0.815 and 0.880 kg/L at 60° C.;
6. has a melting range between 38° and 60° C.;
7. has an acidity/alkalinity of neutral to litmus;
8. exhibits a congealing point of about 63° C.;
9. has a viscosity of 10.7 cSt at 100° C.;s
10. exhibits a UV absorbance at 290 nm of no more than 0.5;
11. has a flash point of 1900° C. (COC); and
12. has a residue on ignition of no more than 0.05%.

47. A method of producing fibrous tissue at or around the G-zone in the vagina of a female patient comprising delivering to the G-zone or tissue surrounding the G-zone in the vagina of a female patient an effective amount of a composition consisting essentially of a mixture of saturated hydrocarbons selected from the group consisting of C₄ to C₆₀ saturated hydrocarbons, said composition exhibiting a viscosity effective for delivery to a predetermined delivery site in said subject to be treated; and allowing said composition to produce a fibrous material at the injection site, resulting in fibrous tissue in said female patient at or around the G-zone.

48. The method according to claim 47 wherein said fibrotic tissue allows the female patient to experience orgasm or sexual pleasure more frequently than without said fibrous tissue.

49. A composition adapted for delivery into selected tissue in a patient consisting essentially of a mixture of saturated hydrocarbons falling within the range of C₄-C₆₀ hydrocarbons having a viscosity effective for delivery into a site in the tissue of a patient, said composition after delivery to said tissue remaining at the site of said delivery without significant migration for a period of at least two days.
